(19) United States
(12) Patent Application Publication
(10) Pub. No.: US 2016/0074628 A1
(43) Pub. Date: Mar. 17, 2016

(54) CATHETER CLIP

(71) Applicant: Cook Medical Technologies LLC, Bloomington, IN (US)

(72) Inventors: Smitha Smith, Winston-Salem, NC (US); Jeremy Thomas, Winston-Salem, NC (US)

(21) Appl. No.: 14/847,410

(22) Filed: Sep. 8, 2015

Related U.S. Application Data
(60) Provisional application No. 62/049,087, filed on Sep. 11, 2014.

(51) Int. Cl. A61M 25/02

(52) U.S. Cl. CPC A61M 25/02; A61M 2025/024; A61M 2025/028

(57) ABSTRACT

A catheter clip for managing a medical device having an elongate catheter shaft is provided. The catheter clip comprises a first body section having a c-shape and forming a first opening configured to accommodate a first portion of medical device. The catheter clip further comprises a second body portion having a u-shape and forming a second opening configured to accommodate a plurality of catheter shaft portions of the medical device therein. An inside surface coincident with the second opening includes a least one indentation configured to accommodate the catheter shaft portion therein, and at least one raised portion adjacent to the indentation and configured to inhibit movement of the catheter shaft portion.

### RELATED APPLICATION

This application claims the benefit of priority from U.S. Provisional Application No. 62/049,087, filed Sep. 11, 2014, and titled “Catheter Clip”, the contents of which is incorporated herein by reference.

### BACKGROUND

The present invention generally relates to a catheter clip. More particularly, the present invention relates to a catheter clip for managing an elongate medical catheter by securing the catheter in a coiled configuration.

Catheters are elongate tubular medical devices for insertion into the body in order to perform a variety of medical procedures. These catheters can vary in size, shape and function, and often consist of a long, flexible, and thin tubular portion that is connected to an accessory or device that a healthcare professional uses to perform the medical procedure. Catheters are generally very fine and delicate in nature, and should be handled, stored, and shipped with great care.

In addition to the delicate nature of catheters, the sterility of catheters is also of paramount importance so as to prevent infection within the body.

During the course of various medical procedures, a healthcare professional often works with a variety of catheter devices. As a function of practicality, given the length of these devices (catheters often range from 170-200 cm long, particularly those used for biliary procedures) and the speedy nature of these procedures, healthcare professionals must balance maintaining the sterility and organization of these lengthy devices with the need to perform the procedure efficiently. In addition, specific catheters may be used more than once during the medical procedure, thereby requiring that the catheter be temporarily stored before being reused. However, catheters can become tangled or dropped, requiring additional time to either untangle the catheter or replaced the contaminated catheter with a brand new sterilized catheter. The length and flexibility of the catheter also poses a risk of injury to the patient, physician or others assisting in the medical procedure.

To manage the catheter, healthcare professionals frequently coil the length of the catheter about itself and then tucking the end of the catheter through the coil. However, the coiled catheter is often unsecure and may inadvertently and unintentionally uncoil itself. In addition, even if the coiled catheter remains coiled, the process of uncoiling the catheter may be time consuming or lead to entanglement, or may cause damage to the catheter.

### BRIEF SUMMARY

One or more embodiments of the present invention provide for an improved catheter management clip that both increases the efficiency of performing the medical procedure while also increasing the ability of healthcare professionals to manage and maintain the sterility of the catheters they are using.

The shape of the catheter clip allows for easy securement of the loops of the catheter, allowing the user to manage the long length of a catheter. The catheter clip can be attached to the body of any medical device or catheter to allow the use of the catheter clip on a wide variety of applications—from endoscopy to vascular applications.

In one embodiment of the invention, a catheter clip for managing a medical device having an elongate catheter shaft is provided. The catheter clip comprises a first body section having a c-shape and forming a first opening oriented in a first direction, the first opening having an interior width that is configured to accommodate a first portion of medical device, the first opening having an exterior width that is less than the width of the interior opening and is configured to inhibit the passage of the first portion of the medical device there through. The catheter clip further comprises a second body portion having a u-shape and including a first elongate portion and a second elongate portion that are spaced apart and generally parallel to each other, the first and second elongate portions forming a second opening oriented in a second direction, the second direction being generally opposite the first direction, the second opening being configured to accommodate a plurality of catheter shaft portions of the medical device therein. At least one of the first and second elongate portion comprises an inside surface coincident with the second opening and having at least one raised portion and at least one indentation adjacent to the at least one raised portion, the at least one indentation configured to accommodate the catheter shaft portion therein, and the at least one raised portion being configured to inhibit transverse movement of the catheter shaft portion.

In another embodiment of the invention, a method of catheter management is provided. The method includes the steps of providing an elongate catheter comprising a proximal portion and a plurality of spaced apart shaft portions; providing a clip comprising a first body portion and a second body portion, wherein said first body portion comprises a first opening and the second body portion comprises a second opening, the second opening having a shape that is different from that of the first opening; attaching the first opening of the first body portion to the proximal portion of the elongate catheter; and disposing the plurality of spaced apart shaft portions within the second opening of the second body portion.

Other systems, methods, features and advantages of the invention will be, or will become, apparent to one with skill in the art upon examination of the following figures and detailed description. It is intended that all such additional systems, methods, features and advantages be within the scope of the invention, and be encompassed by the following claims.

### BRIEF DESCRIPTION OF THE DRAWINGS

The accompanying drawings are included to provide a further understanding of the claims, are incorporated in, and constitute a part of this specification. The detailed description and illustrated examples described serve to explain the principles defined by the claims.

FIG. 1 illustrates a catheter clip with a section securable to a catheter according to an embodiment of the present invention.

FIG. 2 illustrates a catheter clip with a section securable to the handle of a medical device according to an embodiment of the present invention.

FIG. 3 illustrates a catheter clip secured to a catheter according to an embodiment of the present invention.

### DETAILED DESCRIPTION OF THE DRAWINGS AND THE PRESENTLY PREFERRED EMBODIMENTS

The present catheter clip operates to secure the length of a catheter during packaging, in between uses, or for disposal. The catheter clip allows a health professional to manage the length of a catheter by first coiling the catheter and then pushing the coils into the opening of the catheter clip. The health professional may also attach individual coils of the catheter to the catheter clip as the catheter is being coiled. Once engaged, the coils will not detach unless the health professional decides to remove them.

In the present embodiment, the catheter clip has two portions—an attachment portion and a catheter securement portion. The attachment portion allows the anchoring of the catheter clip to either the handle of a medical device or onto the catheter itself The catheter securement portion is the portion the coils of the catheter are pushed into in order to secure the length of the catheter. In one embodiment, this catheter securement portion has a wave-like structure on the inside surface that runs along the length of the legs of the clip. The dipped portions of the wave-like structure contain the coils of the looped catheter to prevent them from detaching. In another embodiment, the catheter clip can be made from any material capable of deforming and later returning to its un-deformed shape to allow for the accommodation of various sizes of coils.

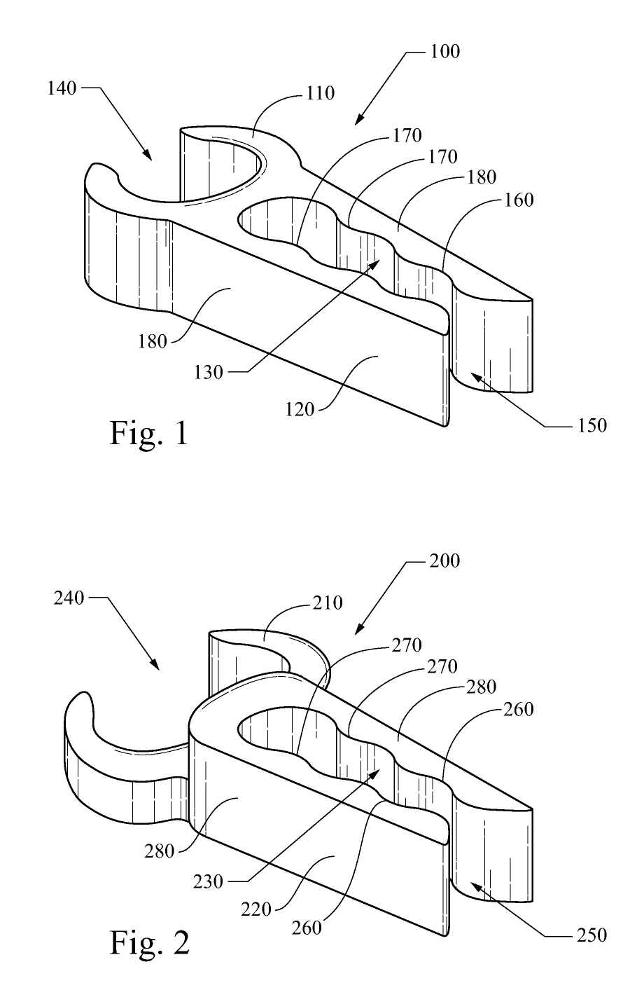

FIGS. 1-3 illustrate various embodiments of the catheter clip used to secure the length of the catheter. FIG. 1 illustrates a first embodiment of the catheter clip 100 having an attachment section 110 and a shaft securement section 120. FIG. 3 illustrates the catheter clip 100 in use and attached to a medical device 300 having a proximal catheter section 310 extending distally from a device handle 320, and an elongate catheter shaft 330 extending distally from the proximal catheter section 310. As will be explained in greater detail below, the attachment section 110 is configured to secure the catheter clip 100 to the proximal catheter section 310, and the shaft securement section 120 is configured to secure one or more coils of the catheter shaft 330.

With reference to FIGS. 1 and 3, the attachment section 110 is the portion of the catheter clip 100 that is configured for attachment to a medical device 300. In the particular embodiment illustrated, the attachment section 110 is configured to engage the proximal catheter section 310 of the medical device 300 so as to anchor or secure the catheter clip 100 thereto. The attachment section 110 comprises a c-shape and forms an attachment opening 140 that accommodates the diameter of the proximal catheter section 310. In one embodiment, the attachment section 110 is composed of a flexible material (such as plastic) capable of deforming and later returning to its un-deformed (original) shape. As a consequence, the exterior portion of the attachment opening 140 can be widened to increase the width or alter the shape thereof to accommodate the diameter of the proximal catheter section 310 when attaching the catheter clip 100 thereto, but will thereafter return to its original shape to secure the catheter clip 100 to the medical device 300. In other words, the attachment opening 140 is configured to allow the attachment section 110 to be attached to the proximal catheter section 310 of the medical device 300 by applying an external force thereto, and then will become secured to the proximal catheter section 310 of the medical device 300 when the external force is removed.

The shaft securement section 120 is the portion of the catheter clip 100 that secures one or more portions of the catheter shaft 330 in a coiled configuration. The shaft securement section 120 comprises a u-shape and includes a pair of flexible legs 180 that form an opening 150 there between. The legs 180 each comprise an inwardly facing surface 130 that defines the opening 150. As best seen in FIG. 3, the opening 150 is configured to receive and secure one or more portions of the catheter shaft 330. Specifically, the catheter shaft 330 of the medical device 300 may be looped and pushed into the opening 150 of the shaft securement section 120, thereby securing the catheter shaft 330 in a coiled configuration. In the particular embodiment illustrated in FIG. 1, the inside surface 130 forms a wave-like structure that is composed of a number of curved indentations or dips 160 and alternating raised portions 170. This wave-like structure can be present on either or both legs 180 of the shaft securement section 120. In the particular embodiment illustrated, wave-like structures are present on the inside surface 130 of both legs 180 and mirror one another. The dips 160 accommodate individual coils of the catheter shaft 330 and the raised portions 170 help contain the individual coils of the catheter shaft 330 within the dips 160. A pair of opposing dips 160 may define an open gap between the inside faces 130 that is approximate the diameter of the catheter shaft 330 to accommodate the catheter shaft 330 therein. A pair of opposing raised portions 170 may define an open gap between the inside faces 130 that is smaller than the diameter of the catheter shaft 330 to inhibit inadvertent movement of the catheter shaft 330 there through. As explained above, this arrangement will tend to maintain the coils of the catheter shaft 330 within the catheter clip 100 unless and until the user desires to uncoil the catheter shaft 330 of the medical device 300.

In the particular embodiment illustrated, the legs 180 of the shaft securement section 120 may be formed so that they are sloped towards each other at the end forming the opening 150. In other words, the inside surfaces 130 may be configured such that the end of the opening 150 that is spaced furthest away from the attachment section 110 is smaller than the end of the opening 150 that is nearest the attachment section 110. Such a configuration will inhibit inadvertent or unintentional removal of the catheter shaft 330 from the shaft securement section 120. However, the shaft securement section 120 is preferably composed of a flexible material capable of deforming and later returning to its un-deformed shape. A flexible material will allow the legs 180 to be flexed apart to increase the width of the opening 150 so as to accommodate the coils of the catheter shaft 330. However, the material must be resilient enough to allow the opening 150 to return to its un-widened state to thereby securing the coils of the catheter shaft 330 within the shaft securement section 120 and prevent inadvertent or unintentional removal therefrom. In the embodiment illustrated, the catheter clip 100 id formed from a plastic material.

In using the catheter clip 100, the user can either first secure the coils of the catheter shaft 330 to the shaft securement section 120, or secure the catheter clip 100 to the proximal catheter section 310 using the attachment section 110. To secure the coils of the medical device 300, the user loops the length of the catheter shaft 330 and pushes the coils of the catheter shaft 330 through the opening 150 of the shaft securement section 120. As discussed above, in this embodiment, the dips 160 and the raised portions 170 of the wave-like structure on the inside surface 130 of the shaft securement section 120 act to contain the coils of the catheter shaft 330 and prevent them from inadvertently or unintentionally detaching. To secure the catheter clip 100 to the medical device 300, the attachment section 110 is pushed onto the proximal catheter section 310. In this embodiment, the attachment opening 140 of the attachment section 110 flexes slightly so as to widen a sufficient distance to accommodate the diameter of the catheter shaft 330. Once the attachment opening 140 of the attachment section 110 has been pushed or disposed over the proximal catheter section 310, the attachment opening 140 returns to its original (unflexed) width to secure the catheter clip 100 to the medical device 300, and thereby secure the coils of the catheter shaft 330 to thereto.

In the illustrated embodiment, the attachment opening 140 of the attachment section 110 and the opening 150 of the shaft securement section 120 are oriented along opposite directions (i.e., 180° apart). However, it should be understood that the openings 140, 150 may be oriented along different paths, such as at a right or acute angle to each other.

Although catheter clip 100 is described as comprising an attachment section 110 and a shaft securement section 120 formed from a flexible material that will “flex” open and close to accommodate various components of the medical device 300, the catheter clip 100 may be formed of a more rigid material and overmolded with a softer or more compliant material such as rubber. The softer material, which is preferably disposed on the inside surfaces of the attachment section 110 and a shaft securement section 120, can be configured to compress to accommodate components of the medical device 300, and then expand to prevent movement thereof. The softer material may also increase the frictional engagement between the contact surfaces of the catheter clip 100 and the medical device 300.

The attachment section 110 and/or the shaft securement section 120 of the catheter clip 100 may also include a closure member (not shown) that will close the ends of openings and further secure the components of the medical device there within. The closure members may comprise a living hinge attached, for example, to one leg 180 of the shaft securement section 120 that allows the closure member to pivot into engagement with the other leg 180 and close the opening 150.

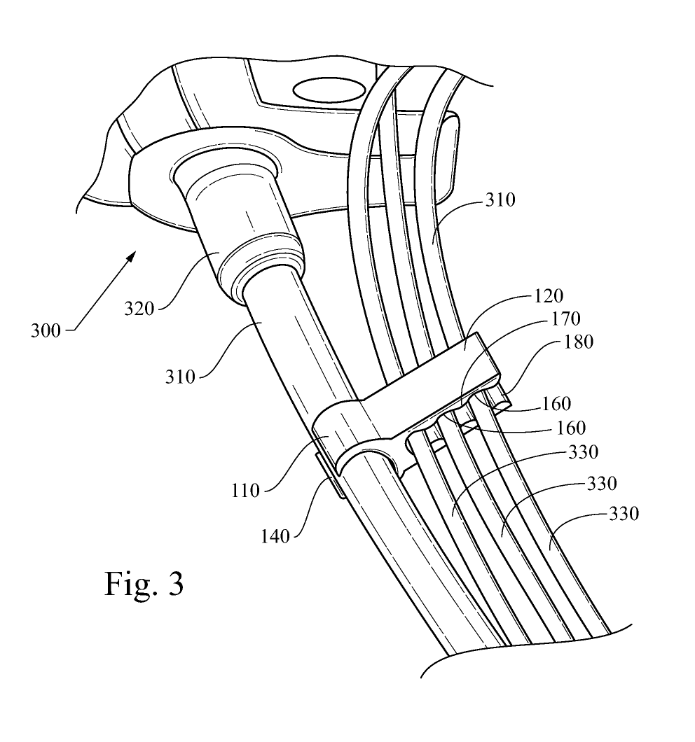

FIG. 2 illustrates another embodiment of the catheter clip 200 for use in securing an elongate medical device 300 in a coiled configuration. In this embodiment, the catheter clip 200 is configured to be connected to the device handle 320 of the medical device 330 (see FIG. 3) and secure the catheter clip 200 thereto. Alternatively, the catheter clip 200 can be configured to attach and secure the catheter clip 200 to other structures such as accessories used in conjunction with the medical procedure, an extension line, a short wire port slide, or any other structure that may be used during the medical procedure.

The catheter clip 200 has two main components, the handle attachment section 210 and the catheter shaft securement section 220. The handle attachment section 210 is the portion of the catheter clip 200 that is attached to the device handle 320. The handle attachment section 210 forms a c-shaped attachment opening 240 that accommodates the diameter or width of the device handle 320. In one embodiment, the handle attachment section 210 may be composed of a flexible material capable of deforming and later returning to its un-deformed shape. The use of a flexible material allows the opposing legs of the handle attachment section 210 to be flexed apart so as to increase the width of the c-shaped attachment opening 240 and accommodate the width or diameter of the device handle 320 therein, but will thereafter return to its original shape. As discussed above, the handle attachment section 210 can be designed to accommodate other structures used in conjunction with the medical procedure.

The catheter shaft securement section 220 is the portion of the catheter clip 200 that secures the looped coils of the medical device 300, and is similar to the shaft securement section 120 of the catheter clip 100 described about. In particular, the catheter shaft securement section 220 has an inside surface 230 that is defined by a pair of flexible legs 280. The legs 280 of the catheter shaft securement section 220 are unattached at one end to form an opening 250 there between. This opening 250 allows for the coils of the catheter shaft 330 to be easily pushed into the catheter shaft securement section 220 and secured there within. As discussed above, in an exemplary embodiment, the inside surface 230 can form a wave-like structure that is composed of a number of alternating dips 260 and raised portions 270. This wave-like structure can be present on either one or both legs 280 of the catheter shaft securement section 220 and can, if present on both legs 280 of the catheter shaft securement section 220, mirror one another. These dips 260 and raised portions 270 serve the same purpose as discussed above in connection with the first embodiment of the catheter clip 100.

The catheter clip 200 is used in the same way as the catheter clip 100. For example, the user can either first secure the coils of the catheter shaft 330 within the looped catheter shaft securement section 220, or secure the catheter clip 200 to the device handle 320 using the handle attachment section 210.

The above-described catheter clips 100, 200 may be incorporated into packaging or a kit that is supplied with the medical device 300. As such, the catheter clips 100, 200 may be packaged with the medical device 300 by being already attached thereto and securing the medical device 300 in a coiled configuration. Alternatively, a variety of catheter clips 100, 200 configured to be used with a variety of medical devices 300 may be packaged and supplied separately.

Although the above-described catheter clips 100, 200 are configured to be removably attached to a medical device 300, the catheter clips 100, 200 may be permanently or otherwise fixedly attached to the medical device 300. For example, the catheter clips 100, 200 may be molded or an integral component of the handle 320 of the medical device 300.

While particular elements, embodiments, and applications of the present invention have been shown and described, it is understood that the invention is not limited thereto because modifications may be made by those skilled in the art, particularly in light of the foregoing teaching. It is therefore contemplated by the appended claims to cover such modifications and incorporate those features which come within the spirit and scope of the invention.

1. A catheter clip for managing a medical device having an elongate catheter shaft, said catheter clip comprising:
a first body section comprising a c-shape and forming a first opening oriented in a first direction, the first opening having an interior width that is configured to accommodate a first portion of medical device, the first opening having an exterior width that is less than the width of the interior opening and is configured to inhibit the passage of the first portion of the medical device there through;
a second body portion comprising a u-shape and including a first elongate portion and a second elongate portion that are spaced apart and parallel to each other, the first and second elongate portions forming a second opening oriented in a second direction, the second direction being opposite the first direction, the second opening being configured to accommodate a plurality of catheter shaft portions of the medical device therein,
wherein at least one of the first and second elongate portion comprises an inside surface coincident with the second opening and having at least one raised portion and at least one indentation adjacent to the at least one raised portion, the at least one indentation configured to accommodate the catheter shaft portion therein, and the at least one raised portion being configured to inhibit movement of the catheter shaft portion.

2. The catheter clip of claim 1 wherein the first elongate portion comprises a first inside surface and the second elongate portion comprises a second inside surface facing the first inside surface, further wherein each of the first and second inside surfaces comprise at least one raised portion and at least one indentation disposed thereon.

3. The catheter clip of claim 2 wherein each of said first inside surface and said second inside surface comprise a plurality of alternating indentations and raised portions, the first inside surface having a profile that mirrors that of the second inside surface.

4. The catheter clip of claim 1, wherein said first opening of said of said first body portion is configured to be secured about one of a handle portion and an enlarged shaft portion of the medical device.

5. The catheter clip of claim 1, wherein said second opening of said second body portion is configured to accommodate and secure at least three catheter shaft portions of the medical device in a side-by-side arrangement.

6. The catheter clip of claim 1 wherein said first inside surface and said second inside surface are each lined with a flexible material configured to enhance frictional contact with the catheter shaft portion of the medical device.

7. The catheter clip of claim 1 wherein said first and second body portions each comprise a flexible material configured to permit a shape of the first and second openings to be altered in response to an externally applied force.

8. The catheter clip of claim 1 wherein said first and second body portions each comprise an interior structure formed from plastic and an exterior surface layer formed from rubber.

9. A system for managing a medical device having an elongate catheter shaft extending from a handle, said system comprising:
a medical device comprising an elongate catheter shaft extending from a handle, the elongate shaft having a proximal shaft portion and a distal shaft portion; and
a catheter clip comprising:
a c-shaped first body section comprising a first opening oriented in a first direction, wherein the first opening is removably attached to the proximal shaft portion of the medical device; and
a u-shaped second body portion comprising a pair of elongate leg portions extending outwardly from the first body section, the pair of elongate leg portions forming a second opening there between, the second opening oriented in a second direction opposite the first direction, wherein the distal shaft portion of the medical device is coiled upon itself and removably disposed within the second opening.

10. The system of claim 9 wherein the pair of elongate leg portions each comprise an inwardly facing surface coincident with the second opening, each inwardly facing surface comprising a wave-like structure comprising a plurality of alternating raised portions and indentations, each of the indentations engaging a spaced apart section of the distal shaft portion.

11. The system of claim 10 wherein each of the alternating raised portions is disposed between adjacent pairs of the spaced apart sections of the distal shaft portion.

12. A method of catheter management, said method comprising:
providing an elongate catheter comprising a proximal portion and a plurality of spaced apart shaft portions;
providing a clip comprising a first body portion and a second body portion, wherein said first body portion comprises a first opening and the second body portion comprises a second opening, the second opening having a shape that is different from that of the first opening;
attaching the first opening of the first body portion to the proximal portion of the elongate catheter; and
disposing the plurality of spaced apart shaft portions within the second opening of the second body portion.

13. The method according to claim 12 further comprising the step of coiling the elongate catheter so as to dispose the plurality of spaced apart shaft portions adjacent to each other.

14. The method according to claim 12 wherein the step of attaching the first opening of the first body portion to the proximal portion of the elongate catheter comprises flexing the first body portion so as to alter the size of the first opening.

15. The method according to claim 12 wherein the step of disposing the plurality of spaced apart shaft portions within the second opening of the second body portion comprises the step of flexing the second body portion so as to alter the size of the second opening.
